(19) United States
(12) Patent Application Publication
(10) Pub. No.: US 2024/0000125 A1
(43) Pub. Date: Jan. 4, 2024

(54) DIET PREPARATION FOR THE NUTRITION OF CANCER PATIENTS WITH A RESTRICTED CALORIE INTAKE

(71) Applicant: FONDAZIONE IRCCS ISTITUTO NAZIONALE DEI TUMORI, Milano (IT)

(72) Inventors: Licia Rivoltini, Milano (IT); Claudio Vernieri, Milano (IT); Filippo Guglielmo De Braud, Milano (IT)

(73) Assignee: FONDAZIONE IRCCS ISTITUTO NAZIONALE DEI TUMORI, Milano (IT)

(21) Appl. No.: 18/257,890

(22) PCT Filed: Dec. 17, 2020
(86) PCT No.: PCT/IT2020/000083

§ 371 Date: Jun. 16, 2023

(51) Int. Cl. A23L 33/00; A23L 33/115; A23L 33/185; A23L 33/21

(52) U.S. Cl. CPC A23L 33/40; A23L 33/115; A23L 33/185; A23L 33/21

(57) ABSTRACT

Daily food preparation for use as a therapeutic adjuvant in the diet of patients suffering from neoplasia, including A) a nutritional combination that contains between 6% and 56% Kcal as carbohydrates, from 7% to 20% Kcal as proteins, from 30% to 86% Kcal as lipids; and B) dietary fiber in an amount between 1 and 30 g, the daily food preparation being characterized by an average caloric content between 199 and 630 Kcal, in which the nutritional combination (A) and the dietary fiber (B) consist of foods of vegetable origin. A further object of the present invention is a kit comprising daily food preparations in accordance with the invention for use as a therapeutic adjuvant in the daily diet of patients suffering from neoplasia.

### FIELD OF THE INVENTION

The present patent application concerns a nutrition plan for patients suffering from different types of neoplasias. It is applicable, on a three-week or four-week basis, before, during and after the administration of different types of anticancer treatments, including radiation therapy, chemotherapy, immunotherapy or endocrine and biological therapies, and also in patients not receiving active anticancer treatments.

### BACKGROUND OF THE ART

The discovery of oncogenes and tumor suppressor genes (TSGs) revealed the biochemical basis of the impact of some metabolic pathways of tumor cells on some peculiar features of tumors, including e.g. uncontrolled proliferation, non-dependence from stimulation of growth factors and resistance to apoptosis.

The metabolism is the assemble of biochemical transformations that take place inside the cells and which include, e.g., the catabolic processes of energy unit production (in the form of ATP or reducing equivalents) and the anabolic processes of complex biomolecule synthesis, such as proteins, lipids and DNA. The metabolic pathway that has been most studied and successfully used in anticancer treatments is the synthesis of nucleotides and deoxynucleotides. Other metabolic pathways have only recently become research focus, based on new discoveries and by virtue of the plateau reached in terms of improving efficacy with traditional therapies.

Similar to highly proliferative healthy cells, cancer cells need a constant supply of ATP and anabolic precursors to support crucial biochemical processes, such as DNA synthesis and repair, protein and lipid synthesis, protein post-translational modifications, formation and re-assembly of membranes and organelles, vesicular transport of intracellular cargo and endocytosis.

It has been observed that many tumors are characterized by reprogramming mechanisms of oncogene-dependent metabolic pathways, e.g., while the mitochondrial oxidation of pyruvate deriving from glucose represents the main source of ATP for healthy proliferative cells, in cancer cells the synthesis of pyruvate is diverted to lactate synthesis metabolism from the mitochondrial oxidation one.

Recent studies have shown that not only glycolysis, but also other metabolic pathways can be regulated differently in cancer cells than in the cells of the corresponding normal tissues. Furthermore, the relationship between oncogenes/TSGs and metabolic reprogramming suggests that the altered regulation of metabolisms crucially contributes to the uncontrolled proliferation of the neoplastic cell.

Therefore, diet changes could have an impact on tumor growth by changing the plasma concentration of different biomolecules and metabolites that support the proliferation and other biological functions of cancer cells. Given the great variety of possible approaches and their unpredictable and complex impact on the multiplicity of blood metabolites, it is not obvious or immediate to be able to design the “ideal” anticancer diet which is safe, well tolerated and accepted by patients and, at the same time, biologically active and effective.

Since moderate chronic calorie restriction significantly reduces tumor incidence in primates and other mammal species, many anti-cancer dietary approaches currently under study are based on the assumption that restriction/modulation of nutrient intake can reduce the cancer cell uptake of metabolites (Vernieri C, Casola S, Foiani M, Pietrantonio F, de Braud F, Longo V. Targeting Cancer Metabolism: Dietary and Pharmacologic Interventions. Cancer Discov. 2016; 6(12):1315-1333. doi:10.1158/2159-8290.CD-16-0615).

### SUMMARY OF THE INVENTION

The Applicant has now identified a specific combination of nutrients, suitable for preparing low-calorie, low-carbohydrate and low-protein meals, useful in patients suffering from different types of neoplasias.

The object of the present invention is a daily food preparation for use as a therapeutic adjuvant in the diet of patients suffering from neoplasia, including
A) a nutritional combination that contains
   carbohydrates having an average caloric content ranging from 6 to 56% based on the total Kcal of the daily food preparation (Kcal/Kcal),
   proteins having an average caloric content ranging from 7 to 20% based on the total Kcal of the daily food preparation (Kcal/Kcal),
   lipids having an average caloric content ranging from 30 to 86% based on the total Kcal of the daily food preparation (Kcal/Kcal); and
B) dietary fibre in an amount ranging from 12 and 30 g,
   the daily food preparation being characterized by an average caloric content between 150 and 650 Kcal, wherein the nutritional combination (A) and the dietary fibre (B) consist of foods of vegetable origin.

A further object of the present invention is a kit comprising food preparations according to claim 1 for use as therapeutic adjuvants in the daily diet of patients affected by neoplasias.

### DETAILED DESCRIPTION OF INVENTION

According to the scope of the present invention, by food preparation (or nutrition plan) is meant a combination of foods suitable for the arrangement of one or more meals.

By daily food preparation, is meant a combination of foods intended to provide sufficient caloric content for one day; in other words, the food preparation of the invention is intended for daily use, as a therapeutic adjuvant in the diet of patients suffering from neoplasias.

By therapeutic adjuvant is meant a biologically active substance or a set of biologically active substances, to be taken in combination with anticancer therapies, to enhance their therapeutic effects. In the present case, such biologically active substances are taken through the diet, with the food preparations of the present invention.

By patients suffering from neoplasias is meant subjects who have been diagnosed by a medical specialist with a malignant tumor of any type, at any stage of growth; examples of neoplasias in subjects suitable for the diet with the food preparation of the invention are breast cancer, colorectal cancer, lung cancer, prostate cancer, melanoma, chronic lymphatic leukemia.

As indicated above, the daily average caloric content of the food preparation is between 199 Kcal and 650 Kcal, preferably between 199 and 630 Kcal. This daily calorie content is low enough to induce specific metabolic effects, including a reduction in the blood concentration of glucose, insulin and insulin-like growth factor (IGF-1), which constitute the metabolic prerequisite for the expected immunomodulatory and anticancer effects to occur.

It should be noted that the maximum calorie content during each diet day is the same for each patient, regardless of weight or other anthropometric parameters (height, body mass index, body composition). The minimum caloric content, on the other hand, is potentially different from patient to patient, as it depends on the ability of each patient to consume the maximum amount of food intended by the schedule or, alternatively, only a part of it.

It should be noted that, according to an embodiment of the invention which is not the subject of the present patent application, the patient suffering from neoplasm can also be subjected to a fasting regime (0 Kcal/day) for a maximum duration of 5 consecutive days.

Generally, by food we mean a substance that, introduced into the animal organism, compensates for its expenditure in living force, provides for the reintegration matter, necessary for any growth, and such factors (proteins, fats, carbohydrates, vitamins, minerals) which are essential for the normal performance of fundamental functions for the individual and the species. In the science of nutrition, simple foodstuff, i.e., proteins, carbohydrates or carbohydrates, lipids or fats, vitamins and minerals, and compound foodstuff, consisting in various proportions of simple foods (milk, meat, fruit, etc.) are distinguished. More generally, by foodstuff is meant food (http://www.treccani.it/vocabolario/alimento/).

In view of the above-mentioned definitions, according to the scope of the invention, by foodstuff is meant a compound food which, for the purposes of carrying out the food preparation of the invention, is of vegetable origin.

By food of vegetable origin is meant a food consisting of vegetables or which is obtained from the vegetable (such as vegetable oils) by chemical/physical treatment of the source plant.

The daily food preparation of the invention comprises a nutritional combination (A) containing
carbohydrates having an average caloric content ranging between 6% and 56%, preferably between 7% and 54%, preferably between 7% and 53% (Kcal/Kcal),
proteins having an average caloric content ranging between 7% and 20%, preferably between 8% and 15%, preferably between 9% and 15% (Kcal/Kcal),
lipids having an average caloric content ranging between 30% and 86%, preferably between 31% and 80%, preferably between 33% and 77% (Kcal/Kcal); and
   dietary fiber (B) in an amount comprised between 1 and 30 g, preferably between 3 g and 25 g, preferably between 6 g and 23 g.

Foodstuffs containing vegetable fiber are included in the daily food preparation as “bulking agents”, which are capable to increase the diet bulk without causing a significant increase in its calorie content.

It should be noted that the percentages of each nutrient (carbohydrates, proteins, lipids) are percentages by weight calculated on the total weight of the nutritional combination (A).

According to a preferred embodiment, at least 78% of the average caloric content of the nutritional combination (A) consists of carbohydrates and lipids. More preferably, the average caloric content provided by the sum of carbohydrates and lipids of the nutritional combination (A) ranges between 78% and 97% (Kcal/Kcal).

The carbohydrates of the daily food preparation of the invention preferably comprise between 0.03% and 0.78% (w/w) by weight of starches; between 0.20% and 1.00% (w/w) by weight of soluble carbohydrates.

The lipids of the daily food preparation preferably comprise between 0.09% and (w/w) by weight of saturated fat; between 0.30% and 0.75% (w/w) by weight of monounsaturated fats; between 0.09% and 0.57% (w/w) by weight of polyunsaturated fats.

The proteins of the daily food preparation preferably consist of 100% vegetable proteins.

The dietary fiber of the daily food preparation preferably comprises between 0 and 1.5 g of soluble fiber; between 0 and 5.5 g of insoluble fiber.

The caloric content of each of the foods contained in the daily food preparation was calculated on the basis of data available on the website www.crea.gove.it (website of “Consiglio per la Ricerca in Agricoltura e per l'analisi dell'economia agraria”), based on the INRAN data set (nut.entreca.it with the software “metaDIETA” (medical Device Software METEDA: www.meteda.it).

The carbohydrates contained in the daily food preparation preferably and mostly originate from bread and fruit.

The proteins of the daily food preparation preferably originate from whole wheat bread, vegetables, plant seeds and combinations thereof.

The lipids of the daily food preparation preferably originate from wholemeal bread, extra virgin olive oil, plant seeds and combinations thereof.

The fibers contained in the daily food preparation preferably originate from whole wheat bread, vegetables, fruit, and combinations thereof.

The daily food preparation of the invention is particularly advantageous as is extremely versatile; in order to improve the efficacy of anticancer therapies, the patient suffering from neoplasia who intends to be on a diet with the preparation of the invention, can make prepare various dishes and choose to consume the foodstuffs both cooked and raw, in liquid form (e.g. smoothies) or in solid form, depending on the preference.

The form in which the patient intends to take the foodstuffs of the preparation, in fact, does not affect the diet biological (immunological or metabolic) effects, which are attributable to the calorie, carbohydrate and protein restriction that characterizes the food preparation of the invention.

Preferably, the daily food preparation of the invention further comprises aqueous drinks in a total amount of between 1.5 L and 3.0 L, more preferably between 2.0 and 2.5 L.

According to a preferred embodiment, the daily food preparation according to the invention provides for the intake of at least 2 liters of liquids per day during the autumn/winter season and at least 2.5 liters of liquids per day during the spring/summer season. The intake of liquids in such quantities is important to ensure adequate hydration, in order to dispose of the organic acids (e.g. acetoacetic acid, beta-hydroxybutyric acid and uric acid) that accumulate in the blood during the diet, and also as a remedy for decreased sense of thirst that can be associated with calorie restriction. In order to stimulate thirst, and therefore to guarantee a sufficient intake of liquids to the patient, the food preparation provides for the possibility of intaking various non-caloric, water-based drinks.

By non-caloric drink is meant a drink having an average caloric content lower than 10 Kcal/liter.

Preferably, the non-caloric aqueous drink is selected from the group consisting of water, green or black tea, non-caloric herbal teas, and combinations thereof.

It should be noted that, despite the calorie content per liter, the daily food preparation allows the intake of coffee (espresso); preferably, the coffee (espresso) is taken in quantities not exceeding two cups a day (about 8 cl, equivalent to about 5 Kcal).

According to a preferred embodiment, the daily food preparation further includes a non-caloric aqueous drink, selected from the group consisting of water, green or black tea, non-caloric herbal teas, and combinations thereof, and/or coffee.

It should be noted that the daily food preparation according to the invention is suitable for use as a therapeutic adjuvant in the diet of subjects suffering from neoplasias for a maximum of 5 consecutive days, and can be repeated on a three-week or four-week basis. Preferably, the daily food preparation is suitable for use as a therapeutic adjuvant in the diet of subjects suffering from neoplasias for a period of between 1 and 5 days, preferably between 2 and 5 days, preferably between 3 and 5 days, preferably between 4 and 5 days, preferably for 5 days.

It should be noted that, according to a preferred embodiment, the daily food preparation contains about 598-630 Kcal during the first day, and about 150-300 Kcal, each day, in the following four (second, third, fourth and fifth) days.

The patients who take the daily food preparation of the present invention are preferably subjects suffering from neoplasias
undergoing a radiotherapy and/or chemotherapy anticancer treatment, or
undergoing an immunotherapeutic anticancer treatment, an endocrine anticancer treatment, or an anticancer treatment with molecularly targeted biological drugs, or
not undergoing any anticancer treatment.

Preferably, in cases of radiotherapy and/or chemotherapy anticancer treatments, non-radio/chemotherapy anticancer treatments, the food preparation according to the invention is taken before (neo-adjuvant), during (co-adjuvant) and after (adjuvant) the administration of the treatment.

More preferably, in the case of anticancer radiotherapy and/or chemotherapy, immunotherapy, endocrine treatment or anticancer treatment with molecularly targeted biological drugs, the food preparation according to the invention is taken starting from two days before the start of the administration of the treatment and until completion of the five-day diet (i.e. up to about 72 hours after starting the anticancer treatment).

For example, in patients receiving intravenous treatments (e.g. chemotherapy, immunotherapy) the diet is started two days before the administration of the treatment, ending on the third day after the start of the administration thereof; in patients receiving oral chemotherapy treatment, the diet is preferably started two days before the start of therapy, and continued until the completion of the five days, regardless of the chemotherapy schedule; in patients receiving radiotherapy treatment, the experimental diet is preferably started two days before the start of radiation therapy, and continued for 5 days; in patients receiving non-chemotherapy oral therapies, the diet is preferably started at the beginning of oral therapy, or during the course thereof, and continued until the completion of the five days.

Preferably, in cases where the patient does not receive any anticancer treatment, the food preparation is administered on a three- or four-week basis for a duration of five consecutive days.

### 1st Embodiment

According to a first embodiment, the daily food preparation of the invention comprises a nutritional combination (A1) comprising:
carbohydrates having an average caloric content between 50% and 56% based on the total average caloric content of the nutritional combination (Kcal/Kcal), preferably between 50% and 54% (Kcal/Kcal), preferably of 52.4% (Kcal/Kcal),
proteins having an average caloric content between 11% and 17% based on the total average total caloric content of the nutritional combination (Kcal/Kcal), preferably between 12% and 15%, preferably of 14.29% (Kcal/Kcal),
lipids having an average caloric content between 31% and 35% based on the total average total caloric content of the nutritional combination (Kcal/Kcal), preferably between 32% and 34%, preferably of 33.03% (Kcal/Kcal);
   dietary fiber (B1) in an amount comprised between 17 g and 29 g, preferably between 19 g and 28 g, preferably between 21 g and 25 g, preferably of 22.53 g, the first embodiment of the daily food preparation being characterized by a average caloric content between 550-630 KCal, in which the nutritional combination (A1) and dietary fiber (B1) consist of foods of vegetable origin.

Preferably, the first embodiment of the daily food preparation is characterized by an average caloric content of about 598 Kcal.

Preferably, such first embodiment is intended for use as an adjuvant for the diet of patients suffering from neoplasia for a period of 1 day.

More preferably, said first embodiment is intended for use as an adjuvant the diet of patients suffering from neoplasia on day one of the diet.

According to a preferred embodiment, at least 82% of the average caloric intake the nutritional combination (A1) consists of carbohydrates and lipids. More preferably, the average caloric intake provided by the sum of carbohydrates and lipids of the nutritional combination (A1) ranges between 82% and 90% (Kcal/Kcal).

The carbohydrates of the daily food preparation of the invention preferably comprise between 0.38% and 0.62% (w/w) by weight of starches; between 0.49% and 0.55% (w/w) by weight of soluble carbohydrates.

The lipids of the daily food preparation preferably comprise between 0.15% and 0.17% (w/w) by weight of saturated fat; between 0.65% and 0.71% (w/w) by weight of monounsaturated fats; between 0.13% and 0.19% (w/w) by weight of polyunsaturated fats.

The dietary fiber (B1) of the daily food preparation preferably comprises between 1.00% and 1.50% (w/w) by weight of soluble fiber; between 5.00% and 5.50% (w/w) by weight of insoluble fiber.

The carbohydrates (carbohydrates) contained in the first embodiment of the daily food preparation preferably originate from bread and fruit.

The proteins of the daily food preparation preferably originate from whole wheat bread, vegetables, and combinations thereof.

The lipids of the daily food preparation preferably originate from wholemeal bread, extra virgin olive oil and combinations thereof.

The fibers contained in the daily food preparation preferably originate from whole wheat bread, vegetables, fruit, and combinations thereof.

Preferably, the daily food preparation of the invention further comprises aqueous drinks in a total amount of between 1.5 L and 3.0 L, more preferably between 2.0 and 2.5 L.

According to a preferred embodiment, the daily food preparation according to the invention provides for the intake of at least 2 liters of liquids during the autumn/winter season and at least 2.5 liters of liquids during the spring/summer season. The daily intake of liquids in such quantities is important to ensure adequate hydration, in order to dispose of the organic acids (e.g. acetoacetic acid and beta-hydroxybutyric) that accumulate in the blood during the diet, and also as a remedy for decreased sense of thirst that can be associated with calorie restriction. In order to stimulate thirst, and therefore to guarantee a sufficient intake of liquids to the patient, the daily food preparation provides for the possibility of intaking non-caloric, water-based drinks.

By non-caloric drink is meant a drink having an average caloric content lower than 10 Kcal/liter.

Preferably, the non-caloric aqueous drink is selected from the group consisting of water, green or black tea, coffee, non-caloric herbal teas, and combinations thereof.

It should be noted that, despite the calorie content per liter, the daily food preparation allows the intake of coffee (espresso); preferably, the coffee (espresso) is taken in quantities not exceeding two cups a day (about 8 cl, equivalent to about 5 Kcal).

### 2nd Embodiment

According to a second embodiment, the daily food preparation of the invention comprises a nutritional combination (A2) containing
carbohydrates having an average caloric content between 6% and 12%, preferably between 7% and 12%, preferably of 8.57% (Kcal/Kcal),
proteins in an amount comprised between 7% and 20%, preferably between 8% and 15%, preferably between 12% and 15%, preferably of 14.90% (Kcal/Kcal),
lipids in an amount comprised between 71% and 86%, preferably between 73% and 80%, preferably between 75% and 80%, preferably of 76.67% (Kcal/Kcal);
   dietary fiber (B2) in an amount comprised between 1 g and 10 g, preferably between 3 g and 8 g, preferably between 4 g and 8 g, preferably of 6.38 g, the second embodiment of the daily food preparation being characterized by a average caloric content between 199 and 310 Kcal, in which the nutritional combination (A2) and dietary fiber (B2) consist of foods of vegetable origin.

Preferably, the daily food preparation according to the second embodiment has an average calorie content of about 256 Kcal.

Preferably, such second embodiment is intended for use as an adjuvant for the diet of patients suffering from neoplasia for a period of time ranging from 2 to 4 consecutive days, more preferably 3 consecutive days.

More preferably, said second embodiment is intended for use as an adjuvant for the diet of patients suffering from neoplasia on the second, third and fourth day of the diet.

According to a preferred embodiment, at least 78% of the average caloric content of the nutritional combination (A2) consists of carbohydrates and lipids. More preferably, the average caloric content provided by the sum of carbohydrates and lipids of the nutritional combination (A2) ranges between 78% and 97% (Kcal/Kcal).

The carbohydrates of the daily food preparation of the invention preferably comprise between 0.03% and 0.78% (w/w) by weight of starches; between 0.22% and 0.97% (w/w) by weight of soluble carbohydrates.

The lipids of the daily food preparation preferably comprise between 0.09% and 0.12% (w/w) by weight of saturated fat; between 0.31% and 0.73% (w/w) by weight of monounsaturated fats; between 0.17% and 0.57% (w/w) by weight of polyunsaturated fats.

The dietary fiber (B2) of the daily food preparation preferably comprises between 0.17 and 0.27 g of soluble fiber; between 1.00 and 5.00 g of insoluble fiber.

The carbohydrates (carbohydrates) contained in the first embodiment of the daily food preparation preferably originate from bread and fruit.

The proteins of the daily food preparation preferably originate from vegetables, dried fruit, and combinations thereof.

The lipids of the daily food preparation preferably originate from extra virgin olive oil, dried fruit, and combinations thereof.

The fibers contained in the daily food preparation preferably originate from whole wheat bread, vegetables, fruit, and combinations thereof.

Preferably, the daily food preparation of the invention further comprises aqueous drinks in a total amount of between 1.5 L and 3.0 L, more preferably between 2.0 and 2.5 L.

According to a preferred embodiment, the daily food preparation according to the invention provides for the intake of at least 2 liters of liquids during the autumn/winter season and at least 2.5 liters of liquids during the spring/summer season. The intake of liquids in such quantities is important to ensure adequate hydration, in order to dispose of the organic acids (e.g. acetoacetic acid and beta-hydroxybutyric) that accumulate in the blood during the diet, and also as a remedy for decreased sense of thirst that can be associated with calorie restriction. In order to stimulate thirst, and therefore to guarantee a sufficient intake of liquids to the patient, the daily food preparation provides for the possibility of intaking non-caloric, water-based drinks.

By non-caloric drink is meant a drink having an average caloric content lower than 10 Kcal/liter.

Preferably, the non-caloric aqueous drink is selected from the group consisting of water, green or black tea, coffee, non-caloric herbal teas, and combinations thereof.

It should be noted that, despite the calorie content per liter, the daily food preparation allows the intake of coffee (espresso); preferably, the coffee (espresso) is taken in quantities not exceeding two cups a day (about 8 cl, equivalent to about 5 Kcal).

### 3rd Embodiment

According to a third embodiment, the daily food preparation of the invention comprises a nutritional combination (A3) containing
carbohydrates for an average caloric content between 33% and 41%, preferably between 35% and 38%, preferably of 36.60% (Kcal/Kcal),
proteins in an amount comprised between 7% and 17%, preferably between 10% and 15%, preferably of 12.40% (Kcal/Kcal),
lipids in an amount comprised between 47% and 55%, preferably between 47% and 53%, preferably of 50.72% (Kcal/Kcal);
   dietary fiber (B3) in an amount comprised between 9 g and 21 g, preferably between 11 g and 18 g, preferably between 13 g and 17 g, preferably of 15 g, the third embodiment of the daily food preparation being characterized by a average caloric content between 320 and 400 KCal, in which the nutritional combination (A3) and dietary fiber (B3) consist of foods of vegetable origin.

Preferably, the daily food preparation according to the second embodiment has an average calorie content of about 365 Kcal.

Preferably, such third embodiment is intended for use as an adjuvant for the diet of patients suffering from neoplasia for a period of 1 day.

More preferably, said third embodiment is intended for use as an adjuvant the diet of patients suffering from neoplasia on day five of the diet.

According to a preferred embodiment, at least 83% of the average caloric content of the nutritional combination (A3) consists of carbohydrates and lipids. More preferably, the average caloric content provided by the sum of carbohydrates and lipids of the nutritional combination (A3) ranges between 83% and 88% (Kcal/Kcal).

The carbohydrates of the daily food preparation of the invention preferably comprise between 0% and 0.22% (w/w) by weight of starches; between 0.78% and 1.00% (w/w) by weight of soluble carbohydrates.

The lipids of the daily food preparation preferably comprise between 0.15% and 0.16% (w/w) by weight of saturated fat; between 0.69% and 0.76% (w/w) by weight of monounsaturated fats; between 0.09% and 0.15% (w/w) by weight of polyunsaturated fats.

The dietary fiber of the daily food preparation preferably comprises between 0 and 0.10 g of soluble fiber; between 0 and 0.10 g of insoluble fiber.

The carbohydrates (carbohydrates) contained in the first embodiment of the daily food preparation preferably originate from bread and fruit.

The proteins of the daily food preparation preferably originate from fruit, vegetables, and combinations thereof.

The lipids of the daily food preparation preferably originate from extra virgin olive oil.

The fibers contained in the daily food preparation preferably originate from vegetables, fruit, and combinations thereof.

Preferably, the daily food preparation of the invention further comprises aqueous drinks in am amount of between 1.5 L and 3.0 L, more preferably between 2.0 and 2.5 L.

According to a preferred embodiment, the daily food preparation according to the invention provides for the intake of at least 2 liters of liquids during the autumn/winter season and at least 2.5 liters of liquids during the spring/summer season. The intake of liquids in such quantities is important to ensure adequate hydration, in order to dispose of the organic acids (e.g. acetoacetic acid and beta-hydroxybutyric) that accumulate in the blood during the diet, and also as a remedy for decreased sense of thirst that can be associated with calorie restriction. In order to stimulate thirst, and therefore to guarantee a sufficient intake of liquids to the patient, the daily food preparation provides for the possibility of intaking non-caloric, water-based drinks.

By non-caloric drink is meant a drink having an average caloric content lower than 10 Kcal/liter.

Preferably, the non-caloric aqueous drink is selected from the group consisting of water, green or black tea, coffee, non-caloric herbal teas, and combinations thereof.

It should be noted that, despite the calorie content per liter, the daily food preparation allows the intake of coffee (espresso); preferably, the coffee is taken in quantities not exceeding two cups a day (about 8 cl, equivalent to 5 Kcal).

### Kit Comprising a Plurality of Daily Food Preparations

A further object of the present invention is a kit comprising daily food preparations according to the invention for use as a therapeutic adjuvant in the daily diet of patients suffering from neoplasias.

Preferably, the kit comprises the first, second and third embodiments of the daily food preparation according to the invention, so as to supply the patient with the food preparations necessary to complete a diet cycle, preferably of 5 consecutive days.

In this sense, what previously discussed in relation to the first, second and third embodiments of the daily food preparation according to the invention applies as it is to the kit.

According to the preferred embodiment, said kit therefore comprises:
a first daily food preparation containing A1): a nutritional combination characterized by an average caloric content ranging from 50% to 56% as carbohydrates (Kcal/Kcal), from 11% to 17% (Kcal/Kcal) as proteins, from 31% and 35% (Kcal/Kcal) as lipids; and B1) dietary fibre in an amount ranging from 19 g to 28 g, the first daily food preparation being characterized by an average caloric content ranging between 550 and 630 Kcal;
a plurality of the second daily food preparation containing: A2) a nutritional combination characterized by an average caloric content ranging between 6% and 12% (Kcal/Kcal) as carbohydrates, from 7% and 20% (Kcal/Kcal) as proteins, from 71% and 86% (Kcal/Kcal) as lipids; and B2) dietary fibre in an amount ranging from 1 g to 10 g, the second daily food preparation being characterized by an average caloric content ranging from 199 Kcal and 310 Kcal; and
a third daily food preparation containing: A3) a nutritional combination characterized by an average caloric content ranging between 33% and 41% (Kcal/Kcal) as carbohydrates, from 7% and 17% (Kcal/Kcal) as proteins, from 47% to 55% (Kcal/Kcal) as lipids; and B3) dietary fibre in an amount ranging from 9 g to 21 g, the third daily food preparation being characterized by an average caloric content ranging from 320 Kcal and 400 Kcal;
   wherein the components of the nutritional combinations (A1, A2, A3) and the dietary fibre (B1, B2, B3) are foods of vegetable origin.

The use of the daily food preparation according to the invention is associated with a series of significant immunoregulatory effects targeting the peripheral blood of the patients. These effects include:
an important reduction in the percentage of monocyte subpopulations with immunosuppressive function, and cytokines responsible for their mobilization from the bone marrow
a simultaneous increase in the percentage of activated anticancer lymphocyte subpopulations, which in various studies have shown to correlate with a better response of patients to standard anticancer treatments.

These immunoregulatory effects had never been previously described in patients suffering from different types of neoplasias. Our recent discoveries show that the experimental diet is able to stimulate the activation of anticancer immunity in patients suffering from different types of neoplasias, treated with different types of concomitant anticancer therapies.

### EXAMPLES

For illustrative and non-limiting purposes, below are examples of the daily food preparation and the kit according to the invention.

### Example 1: Experimental Diet in a Patient with Breast Cancer and Treated with Concomitant Chemotherapy

day 1: Breakfast: a cup of green tea. Lunch: 100 gr of cauliflower, 90 gr of broccoli, 70 gr of mixed salad and 20 gr of fennel, seasoned with salt and a spoonful of extra virgin olive oil (12 g), plus 100 gr of wholemeal bread. Dinner: green salad and one 150 g apple. About 2 liters of water

day 2: Breakfast: 1 cup of green tea. Lunch: 100 gr of cauliflower+100 gr of broccoli, seasoned with teaspoon of extra virgin olive oil (6 g) and ¼ of lemon; 1 no-sugar coffee. Dinner: 12 gr of nuts.

day 3: Breakfast: 1 cup of black tea. Lunch: 200 gr of Brussels sprouts with a teaspoon of extra virgin olive oil (6 g). Dinner: 15 gr of almonds. Also 2 liters of water during the day.

day 4: Breakfast: 1 cup of black tea. Lunch: 200 gr of cauliflower and Brussels sprouts seasoned with teaspoon of extra virgin olive oil (6 g). Dinner: 80 gr of green salad with 12 gr of walnuts.

day 5: Breakfast: 1 green tea. Lunch: 300 gr of spinach, broccoli, fennel, salad, seasoned with a spoonful of extra virgin olive oil (12 g)+half of a 200 g apple (100 g in total). Dinner: 100 gr of green salad seasoned with teaspoon of oil+half of a 200 g apple (100 g in total).

### Example 2: Experimental Diet in a Patient with Lung Cancer not Treated with Other Concomitant Anticancer Treatments

day 1: Breakfast: nothing. Lunch: 200 g of salad+40 g of onions+1 tablespoon of extra virgin olive oil (12 g)+70 g of whole wheat bread. Dinner: 100 g of salad+1 teaspoon of olive oil (6 g)+one 200 g apple

day 2: Breakfast: 2 herbal teas. Lunch: 120 g of salad+20 g of fennel+1 teaspoon of extra virgin olive oil (6 g). Dinner: 25 g of shelled walnuts.

day 3: Breakfast: 2 non-caloric herbal teas (ginger+chili pepper). Lunch: 150 g of cabbage+1 teaspoon of extra virgin olive oil (5 g). Dinner: 20 g of shelled walnuts

day 4: Breakfast: 2 non-caloric herbal teas. Lunch: 100 g of fennel+1 teaspoon of extra virgin olive oil (5 g). Dinner: 20 g of shelled walnuts.

day 5: Breakfast: 2 non-caloric herbal teas. Lunch: 180 g of spinach+1 tablespoon of extra virgin olive oil (5 g). Dinner: 130 g of salad+1 teaspoon of extra virgin olive oil (5 g).

### Example 3: Experimental Diet in a Patient with Breast Cancer and Treated with Concomitant Chemotherapy

Day 1: Breakfast: one cup of green tea. Lunch: 300 g among spinach, cauliflower, cabbage, lettuce, broccoli, courgettes dressed with salt and a spoon of extra virgin olive oil (12 g), 100 g of whole wheat bread. Dinner: green salad (150 g) dressed with 5 g of extra virgin olive oil and a fruit among apple, pear, orange (250 g). About 2 L of water.

Days 2-4: Breakfast: 1 cup of green tea. Lunch: 200 g among green salad, spinach, cabbage, cauliflower, broccoli, lettuce, courgettes or 50 g of onion, dressed with a tea spoon of extra virgin olive oil (5 g). Dinner: 20 g of shelled walnuts or 30 g of almonds. About 2 L of water.

Day 5: Breakfast: 1 cup of green tea. Lunch: 300 g among spinach, cabbage, cauliflower, broccoli, lettuce, courgettes, dressed with a tea spoon of extra virgin olive oil (12 g) and a fruit among apple, orange, pear of 250 g. Dinner: 100 g green salad dressed with a tea spoon of oil (5 g). About 2 L of water.

1.-11. (canceled)

12. A dietary method for enhancing the effect of anticancer treatments in patients suffering from neoplasia, said method comprising:
administering a daily food preparation including:
A) a nutritional combination characterized by an average caloric content
ranging from 6% to 56% (Kcal/Kcal) as carbohydrates;
ranging from 7% to 20% (Kcal/Kcal) as proteins;
ranging from 30% to 86% (Kcal/Kcal) as lipids; and
B) dietary fiber in an amount ranging from 1 to 30 g,
the daily food preparation being characterized by an average total caloric content between 199 and 650 Kcal/day, wherein the nutritional combination (A) and the dietary fiber (B) consists of foods of vegetable origin.

13. The dietary method according to claim 12, wherein at least 78% (Kcal/Kcal) of the average caloric content of the nutritional combination (A) consists of carbohydrates and lipids.

14. The dietary method according to claim 12, wherein the carbohydrates comprise between 0.03% and 0.78% (w/w) by weight of starches and between 0.20% and 1.00% (w/w) by weight of soluble carbohydrates.

15. The dietary method according to claim 12, wherein the lipids comprise between 0.09% and 0.17% (w/w) by weight of saturated fats; between 0.30% and 0.73% (w/w) by weight of monounsaturated fats; between 0.09% and 0.57% (w/w) by weight of polyunsaturated fats.

16. The dietary method according to claim 12, wherein the dietary fiber comprises between 0 and 1.5 g of soluble fiber and between 0 and 5.5 of insoluble fiber.

17. The dietary method according to claim 12, wherein the foods of vegetable origin are raw or cooked foods.

18. The dietary method according to claim 12, further comprising a non-caloric aqueous drink and/or coffee in an amount ranging from 1.5 to 2.5 L.

19. The dietary method according to claim 12, wherein the patients suffering from neoplasia are undergoing a radiotherapy and a chemotherapy anticancer treatment, or
a radiotherapy anticancer treatment; or
a chemotherapy anticancer treatment, or
an immunotherapy anticancer treatment, an endocrine anticancer treatment, or an anticancer treatment, or an anticancer treatment with molecularly targeted biological drugs.

20. The dietary method according to claim 12, comprising the daily administration of the daily food preparation in patients suffering from neoplasia for a maximum of 5 consecutive days.

21. Anticancer-adjuvant dietary kit comprising
one unit of a first daily food preparation containing: A1) a nutritional combination characterized by an average caloric content ranging from 50% to 56% (Kcal/Kcal) as carbohydrates, from 11% and 17% (Kcal/Kcal) as proteins, from 31% to 35% (Kcal/Kcal) as lipids; and B1) dietary fiber in an amount ranging from 19 g to 28 g, the first preparation being characterized by an average total caloric content of between 550 and 630 Kcal/day;
a plurality of a second daily food preparation containing: A2) a nutritional combination characterized by an average caloric content ranging from 6% to 12% (Kcal/Kcal) as carbohydrates, from 7% to 20% (Kcal/Kcal) as proteins, from 71% to 86% (Kcal/Kcal) as lipids; and B2) dietary fiber in an amount ranging from 1 g to 10 g, the second preparation being characterized by an average caloric content of between 199 and 310 Kcal/day; and
one unit of a third daily food preparation containing A3): a nutritional combination characterized by an average caloric content ranging from 33% to 41% (Kcal/Kcal) as carbohydrates, from 7% to 17% (Kcal/Kcal) as proteins, from 47% to 55% (Kcal/Kcal) as lipids; and B3) dietary fiber in an amount ranging from 9 g to 21 g, the third preparation being characterized by an average caloric content of between 320 and 400 Kcal/day;
wherein the components of the nutritional combinations (A1, A2, A3) and the dietary fiber (B1, B2, B3) are foods of vegetable origin.

22. A therapeutic method for enhancing the effect of anticancer treatments in patients suffering from neoplasia comprising
the administration of a daily food preparation including
A) a nutritional combination characterized by an average caloric content
ranging from 6% to 56% (Kcal/Kcal) as carbohydrates,
ranging from 7% to 20% (Kcal/Kcal) as proteins,
ranging from 30% to 86% (Kcal/Kcal) as lipids; and
B) dietary fiber in an amount ranging from 1 and 30 g,
the daily food preparation being characterized by an average total caloric content between 199 and 650 Kcal/day, wherein the nutritional combination (A) and the dietary fiber (B) consist of foods of vegetable origin.
